SEQUENCE LISTING (1 sequence)

SEQ ID NO: 1 - protein, 279 aa, Escherichia coli
MATHKPINILEAFAAAPPPLDYVLPNMVAGTVGALVSPGGAGKSMLALQLAAQIAGGPDLLEVGELPTGPVIYLPAEDPPTAIHHRLHALGAHLSAEERQAVADGLLIQPLIGSLPNIMAPEWFDGLKRAAEGRRLMVLDTLRRFHIEEENASGPMAQVIGRMEAIAADTGCSIVFLHHASKGAAMMGAGDQQQASRGSSVLVDNIRWQSYLSSMTSAEAEEWGVDDDQRRFFVRFGVSKANYGAPFADRWFRRHDGGVLKPAVLERQRKSKGVPRGEA

(19) United States
(12) Patent Application Publication
(10) Pub. No.: US 2004/0116659 A1
(43) Pub. Date: Jun. 17, 2004

(54) Helicase inhibitors

(76) Inventor: Wolfram Saenger, Berlin (DE)

Correspondence Address: BIRCH STEWART KOLASCH & BIRCH, PO BOX 747, FALLS CHURCH, VA, 22040-0747, US

(21) Appl. No.: 10/240,407

(22) PCT Filed: Mar. 30, 2001
(86) PCT No.: PCT/EP01/03659

(30) Foreign Application Priority Data
Mar. 31, 2000 (EP) 00106927.7

(51) Int. Cl. G06F 19/00; G01N 31/00; C07K 14/47

(52) U.S. Cl. 530/350

(57) ABSTRACT

The present invention relates to a crystal of a hexamer of a bacterial helicase, preferably the RepA helicase as well as complexes thereof useful in the development of antibiotics. The invention furthermore relates to methods of identifying inhibitors of said bacterial helicases. The present invention is based on the elucidation of the three-dimensional structure of the RapA helicase by crystallographic means.

The specification recites a number of prior art documents. The disclosure content of these prior art documents is incorporated into the specification by reference.

The present invention relates to a crystal of a hexameric bacterial helicase, preferably the RepA helicase as well as complexes thereof useful in the development of antibiotics. The invention furthermore relates to methods of identifying inhibitors of said bacterial helicases. The present invention is based on the elucidation of the three-dimensional structure of the RepA helicase by crystallographic means.

The helicases are ubiquitous enzymes required for DNA replication, recombination, transcription and repair (Matson and Kaiser-Rogers, 1990; Lohman and Bjornson, 1996) and for RNA translation, splicing of mRNA and assembly of ribosomes (Luking et al., 1998); malfunction of specific helicases results in human diseases like Bloom's and Werner's syndromes (Ellis, 1997) and has been associated with the development of cancer (Egelman, 1996) and with aging (Bowles, 1998). Helicases engaged in DNA replication are ring-shaped oligomers that require a loader protein or single-stranded DNA (ssDNA) regions to initiate unwinding of double stranded DNA (dsDNA) at the replication fork. DNA unwinding is strictly processive (vectorial) in 5′→3′ or 3′→5′ direction and driven by hydrolysis of nucleoside triphosphates (NTPs). The two unwound ssDNA strands serve as templates for DNA polymerase in the synthesis of new, complementary DNA. On the leading strand, DNA synthesis occurs directly but on the lagging strand, short RNA primers are synthesized by primase and extended by DNA polymerase to form Okazaki fragments that are later ligated into DNA. Besides these proteins, others are required for DNA replication, all orchestrated in their work by the action of helicase.

Comparison of amino acid sequences of a large variety of DNA and RNA helicases suggests two large superfamilies, SF1 and SF2, with seven loosely conserved motifs (Gorbalenya and Koonin, 1993). The additional smaller family of DnaB-like helicases shows five conserved motifs H1, H1A, H2, H3, H4 (Ilyina et al., 1992). Among all helicases, only the Walker A- and B-motifs (Walker et al., 1982; Schulz, 1992) are shared as these are involved in ATP hydrolysis; they correspond to H1 and H2 of the helicase family with five conserved motifs. Thus far, no high resolution X-ray structure of an intact hexameric helicase has been published. Structures are known of the dimeric DNA helicases B. stearothermophilus PcrA (Subramanya et al., 1996; Valenkar et al. 1999), and E. coli Rep (Korolev et al., 1997), and of the monomeric RNA helicase domain from hepatitis C virus, NS3 (Yao et al., 1997); they all belong to the family with seven conserved motifs. Of the hexameric helicases, only truncated domain structures of the DnaB family are known: the N-terminal domain of hexameric E. coli DnaB (Fass et al., 1999) and the helicase domain of the bacteriophage T7 helicase-primase (Sawaya et al., 1999), which are representatives of the DnaB-like helicase family with five conserved motifs. Of the intact, unmodified replicative helicases with 6 or possibly 12 subunits, however, only electron microscopic studies have been published, summarized in (Egelman, 1996; Bárcena, 1998). They are all of low resolution and do not show atomic details that are necessary for understanding the functional mechanisms of these enzymes. In several papers discussed in (Bird et al., 1998), the structural similarity between the DNA strand exchange factor E. coli RecA (Story et al., 1992) and F1-ATPase (Abrahams et al., 1994) was extrapolated to hexameric helicases. It was suggested that these helicases should be structurally homologous, with RecA-like ATP binding domains organized into hexameric, annular structures.

The structural analyses effected so far for hexameric bacterial helicases are, however, not sufficient for devising specific inhibitors of these enzymes. Rather, the elucidation of a first three-dimensional structure of the family of said helicases is required in order to develop inhibitors that may be directly used as antibiotics or may give rise to lead compounds useful in the development of antibiotics. Consequently, the technical problem underlying the present invention was to provide such a three-dimensional structure. The solution to said technical problem is achieved by providing the embodiments characterized in the claims.

Thus, the present invention relates to a crystal of a hexamer of a protein consisting of the amino acid sequence of SEQ ID NO: 1, said crystal being obtainable by (a) disrupting E. coli cells comprising a multicopy plasmid encoding Rep A helicase, (b) removing the cell debris of the disrupted E. coli cells, (c) applying the supernatant of said disrupted E. coli cells to an ion exchange column, (d) eluting said polypeptide protein hexamer with a salt gradient of buffer systems A (20 mM Tris-HCl, pH 8.0, 10% glycerol, 0.1 mM EDTA, 50 mM NaCl) and B (20 mM Tris-HCl, pH 8.0, 10% glycerol, 0.1 mM EDTA, 2 mM NaCl), (e) dialyzing the eluted protein hexamer against buffer A, (f) applying the dialyzed protein hexamer to a heparin-agarose column, (g) eluting the protein hexamer with a gradient of buffer systems A and B as recited in step (d), (h) applying the eluted protein hexamer to a gel filtration column, (i) concentrating the protein hexamer to 10 mg ml⁻¹ in 20 mM Tris-HCl, pH 8.0, 10% glycerol, 0.1 mM EDTA and 150 mM NaCl, (j) mixing 3 μl of concentrated protein hexamer as obtained in step (i) with 3 μl of a reservoir solution containing 20-22% (w/v) polyethyleneglycol monomethylether 5000, 2-5% (w/v) 2-methyl-2,4-pentanediol, 0.1 M citrate buffer, pH 6.0, (k) growing crystals by (1) transferring said mixture obtained in (j) to a siliconized microscope cover slip, (2) turning said microscope cover slip upside down, (3) slightly pressing said cover slip on the greased top of a well in a LINBRO® plate containing 1 ml of the reservoir solution, such that a closed system is formed in which the protein hexamer crystallizes; and (I) soaking the crystals obtained in step (k) for 24 hours in said reservoir solution containing 1 mM of ortho-chloromercurinitrophenol.

When referring to the crystal of the invention throughout this specification, it is implicitly also referred to the structure of said crystal.

The amino acid sequence of SEQ ID NO: 1 is available from the database Swiss-Prot under accession number P 20356.

The present invention also relates to a crystal of a hexamer of a protein consisting of the amino acid sequence of SEQ ID NO: 1, wherein said crystal belongs to the monoclinic space group P2₁ with unit cell constants a=104.4 Å, b=179.2 Å, c=116.3 Å, α=90.0°, β=108.8°, γ=90.0°. The crystal obtainable by the above referenced method bears the physiochemical properties referred to here. This means that the crystal shows an internal symmetry of a two-fold screw axis and the unit cell constants are unique so that they can serve as fingerprints to characterize the crystal. These data were obtained from X-ray diffraction patterns of the crystal.

The technical problem underlying the present invention was solved by generating crystals of RepA helicase and determining its three-dimensional structure by X-ray methods employing synchrotron radiation. The hexameric replicative DNA helicase RepA is encoded by the 8684 bp plasmid RSF1010 which may be found in almost all Gram-negative bacteria (Scherzinger et al., 1991) and confers resistance to streptomycin and sulfonamides. RepA is one of the smallest known hexameric helicases with 278 amino acids of sequence SEQ ID NO: 1 in the monomer, and a molecular weight of 29.8 kDa; it unwinds DNA in 5′-3′ direction and is biochemically well characterized (Scherzinger et al., 1997).

The crystallization of the RepA protein has been reported (Röleke et al., 1997). Similarly, the protein has been biochemically characterized (Scherzinger et al., 1997). Yet, the elucidation of the three-dimensional structure by X-ray crystallography has remained elusive so far. In particular, conventional approaches of growing crystals useful for such a three-dimensional analysis have failed in the past. Only by providing a new approach using a suitable heavy atom derivative, the well-known phase problem of X-ray crystallography process could be overcome. One of the particular problems associated with the application of conventional methods was in the present case that the crystals comprising the heavy atoms usually did not display the same unit cell constants as the native crystals. Accordingly, those crystals could not be used for further analysis because of their non-isomorphic structure. Another problem arose from the fact that Cys172 which was expected to yield a suitable heavy atom derivative by soaking crystals with such compounds did not react so that no heavy atom derivative was obtained. Only by mixing a large number of heavy atom compounds with bacterial helicase RepA and then subjecting these samples to mass spectroscopic analysis, a mercury compound could be identified that gave rise to a heavy atom derivative useful for the solution of the phase problem. As a single heavy atom derivative is usually not sufficient to determine the three-dimensional structure of a molecule as complex as hexameric RepA, the novel method of multi-wavelength anomalous dispersion (MAD) was employed using synchrotron radiation that relies on X-radiation at certain well-defined wavelengths and is only obtainable from synchrotrons and not from the common in-house X-ray radiation sources.

The crystal of the invention is the first crystal revealing the complete three-dimensional structure of a hexameric bacterial helicase. This crystal structure may now be used for overcoming the problems of the prior art, namely to select for specific inhibitors of said helicase which do not interfere with the function of eukaryotic helicases, in particular mammalian/human helicases. The crystal of the invention may be prepared according to the setup of steps identified above. However, the invention also comprises crystals that are prepared by other methods as long as these crystals have the same three-dimensional structure as the crystal identified herein above. In general, the crystal of the invention may be prepared according to the following typical protocol: (a) concentrating an essentially pure RepA helicase to about 10 mg/ml in a buffer; (b) for crystallization, mixing concentrated helicase solution with a suitable amount of reservoir solution (batch method); mixing concentrated helicase solution with a reservoir solution and equilibrating the resulting mixture against the reservoir solution (vapor diffusion method); or dialyzing concentrated helicase solution against a reservoir solution (microdialysis method); and (c) soaking the crystals obtained in (b) in a reservoir solution containing ortho-chloromercurinitrophenol.

The purification of protein used for concentration can be done according to conventional protocols. Typically, a microorganism is transformed with an expression vector containing one or more copies of the gene encoding RepA helicase. The microorganisms, typically bacteria, are grown under conditions that allow an optimized expression of the helicase. Possible regulatory elements permitting expression in prokaryotic host cells comprise, e.g., the PL, lac, trp or tac promoter in E. coli. Beside elements which are responsible for the initiation of transcription such regulatory elements may also comprise transcription termination signals, such as the SV40-poly-A site or the tk-poly-A site, downstream of the nucleic acid molecule. Furthermore, depending on the expression system used leader sequences capable of directing the polypeptide to a cellular compartment or secreting it into the medium may be added to the coding sequence of the nucleic acid molecule of the invention and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including a C- or N-terminal identification peptide imparting desired characteristics, e.g., stabilization or simplified purification of expressed recombinant product. If the helicase is exported into the medium, it can conveniently be purified from the culture supernatant. If the expressed helicase is maintained within the host cell, then said host is disrupted as a first step of the purification process. After recovery of a crude mixture from the cell culture, the helicase may be purified by using a combination of various purification steps. Some of these purification steps may be repeated, if desired. The order of steps is not prescribed. The purification steps should include the removal of cell debris, preferably by centrifugation. The helicase present in the cell culture supernatant is advantageously purified by employing a method comprising an affinity purification step.

Such an affinity purification step may make use of compounds that bind to proteins interacting with DNA such as heparin agarose. Alternatively, for example, antibodies raised against the helicase may be used for the purification process wherein said antibodies (or fragments thereof) are preferentially coupled to a column material. Antibodies against helicase RepA may be obtained by conventional immunization protocols such as described, for example, in Harlowe and Lane, “Antibodies, A Laboratory Manual”, CHS Press, 1988, Cold Spring Harbor. This purification step may be combined with gel filtration and anion exchange chromatography steps which may be effected, either alone or in combination, before or after the affinity purification step. Elution from the binding material is effected by using suitable buffers (salt gradients). Protocols for the elution can be adapted by the person skilled in the art on the basis of his/her common knowledge. The purification protocol may further comprise one or more dialysis steps. Preferred buffers for any of the above recited steps are provided in connection with the main embodiment of the invention. The order of purification steps is also preferably effected as described in the first embodiment recited herein above. The growth of crystals is preferably effected by the method described in Röleke (loc. cit.). A preferred embodiment of the step of growing crystals is also provided in connection with the main embodiment of this invention referred to herein above. Each of the steps identified in this protocol may be further and separately refined, as specified herein above. In addition, the invention also comprises crystals that are prepared of the other hexameric helicases as long as these have a three-dimensional structure comparable to that identified herein above.

Employing the data obtainable with the crystal of the invention, new drugs effective in the treatment of infectious diseases conferred by gram-negative and gram-positive bacteria may be generated. The three-dimensional structure of the helicase allows to identify points of contact with the co-factor ATP as well as with the nucleic acid substrate. On the basis of the three-dimensional structure, it was concluded in accordance with the present invention that amino acids Lys43, Glu77, Asp140 and His179 would be catalytically active in RepA helicase. In addition, Arg207 is expected to play the role of an “arginine finger” as it is located in the ATP-binding active site of one subunit but is part of the adjacent subunit in the RepA hexamer. In one alternative, potential blocking agents to these catalytically active amino acids may be tested for and simultaneously be tested on eukaryotic helicases. Only those agents that block the bacterial helicases but not the eukaryotic helicases, are to be formulated into pharmaceutical compositions or used as lead compounds for the downstream development of blocking agents suitable for formulation into pharmaceutical compositions. In a further alternative, co-crystallization of substrate (e.g. ATP and non-hydrolyzable analogs and/or ssDNA) and helicase will allow to determine specific changes in the three-dimensional structure when an interaction with a substrate takes place. Inhibitors of such a structural change may be devised on the basis of a comparison between the referenced crystals. A specific alternative arising from the above for developing or identifying inhibitors takes into account that the referenced bacterial helicase of hexameric structure has an ATP binding site constituted by two different subunits, in contrast to nearly all known eukaryotic helicases wherein the active center is contained within one subunit.

The invention also relates to a crystal of a further hexameric bacterial helicase, the structure of said crystal being derived from the crystal structure of the invention by employing the three-dimensional structure of RepA as a search model to locate said further helicase of comparable structure to RepA in its own crystal unit cell by rotation and translation searches.

If a hexameric helicase with homologous structure to RepA has been crystallized and X-ray data have been measured, the above mentioned phase problem of crystallography can be solved by the “molecular replacement” method. In this method, the three-dimensional structure of RepA is used as a search model to locate the unknown helicase of comparable structure to RepA in its own crystal unit cell by rotation and translation searches. This avoids the cumbersome search for heavy atom derivatives.

The method of “molecular replacement” is applicable if the crystal structure of one protein (A) is known and if, crystals of a homologous protein (B) have been obtained. X-ray diffraction data of crystals of protein (B) are collected and the three-dimensional model of protein (A) is used to search for its orientation (“rotation”) and position (“translation”) in the crystal unit cell or protein (B), utilizing the X-ray diffraction data of the latter. If the search has been successful as indicated by certain correlation coefficients, the model of protein (A) is refined against the X-ray data of the homologous protein (B) until convergence is achieved as indicated by the crystallographic (reliability) R-factor. As a result, the three-dimensional structure of protein (B) is obtained.

The provision of the crystal and the crystal structure of the bacterial RepA helicase is a major breakthrough for the development of antibiotics. Now that the first three-dimensional structure of a hexameric helicase has been identified, said structure can be used for determining crystal structures of further bacterial helicases belonging to this family. It is important to note that the determination of such crystal structures is possible for the person skilled in the art without further ado. Simply, the person skilled in the art can apply the molecular replacement methodology referred to above to determine the three-dimensional crystal structure of further hexameric bacterial helicases.

Additionally, the present invention relates to a crystal comprising a hexamer of a protein of the invention and a ligand or ligands. These ligands are preferably helicase substrates and thus can be nucleoside triphosphates or analogues-thereof, or ssDNA oligonucleotides or analogues thereof, the known inhibitor heliquinomycin and analogues thereof. Ligand(s) can further be any other molecule showing affinity to RepA. The crystal of the invention comprises the helicase and the ligand(s) in complexed form. The crystal of the invention can conventionally be obtained by following either of two routes: First, helicase and ligand(s) may be co-crystallized. Second, the ligand(s) may enter the crystallized helicase by diffusion and then form a complex therewith.

The term “helicase substrate” is intended to mean any compound that is processed by a bacterial helicase of hexameric structure. Such a compound may be of natural or of (semi)synthetic origin. Said substrate may be subject to the ATPase activity of the helicase or to its DNA-unwinding activity. If the helicase substrate is subject to the ATP cleaving activity, then said activity may be assessed by measuring the release in organic phosphate with time. In the case that said substrate is subject to the helicase activity of the enzyme, then this activity may be measured by the amount of single-stranded DNA that is released with time from a double-stranded DNA substrate. Convenient means for measuring the amount of single-stranded DNA is applying the substrate to polyacrylamide gel electrophoresis and determining whether and how much single-stranded DNA is detectable on a gel.

The term “analogue of a nucleoside triphosphate”, in connection with the present invention is intended to mean the non-hydrolizable derivatve described below or derivatives where the base, ribose or triphosphate are substituted by related but chemically modified moieties.

Crystals comprising complexes of the present invention can be produced along the protocol that was employed for generating the crystals of the hexameric helicase of the invention. The crystal comprising the complex of the present invention will be particularly useful for the identification and development of inhibitors, as has been indicated herein above.

In a more preferred embodiment of the crystal of the invention, said ligand is a helicase substrate.

In a even more preferred embodiment of the crystal of the invention, said helicase substrate is a nucleoside triphosphate or an analogue thereof.

In a particularly preferred embodiment of the crystal of the invention, said analogue is a non-cleavable analogue.

The term “non-cleavable” bears the meaning that an enzyme cleaving a substrate to yield products cannot cleave a substrate analogue in which the scissile bond is replaced by a bond that resists cleavage. If such a substrate analogue binds to the active site of its enzyme, the latter is inhibited as the cleavage reaction cannot occur and products are not formed.

In a most preferred embodiment, said non-cleavable analogue is a non-cleavable ATP-analogue, i.e. an analogue that cannot be cleaved into APD and inorganic phosphate or AMP and pyrophosphate

In a further particularly preferred embodiment of the invention, said non-cleavable ATP-analogue is ATPγS, AMPPNP, AMPPCH₂P, or ADP [AlF₄]⁻ or an analogue wherein adenine is replaced by guanine, cytosine, thymine or another heterocycle binding to said protein hexamer.

In an additional particularly preferred embodiment of the invention, the ribose moiety of said non-cleavable analogue is replaced by another cyclic or non-cyclic moiety.

In another preferred embodiment of the crystal of the invention comprising the complex, said helicase substrate is a single-stranded DNA or a double-stranded DNA with a single-stranded overhang or an analogue thereof or heliquinomycin. The binding of ssDNA to helicase can be monitored by measuring the ATPase activity which is significantly stimulated in the presence of ssDNA.

In a most preferred embodiment of the crystal of the present invention, said single-stranded DNA is a DNA oligonucleotide. The composition or sequence of said oligonucleotide are not essential as helicases are non-specific. However, said oligonucleotide must have a minimal length, which is a decanucleotide for RepA. The same or a very similar length is expected to be the minimal length for other hexameric helicases.

Furthermore, the present invention relates to a method of identifying an inhibitor of a bacterial helicase of hexameric structure comprising (a) determining the three-dimensional conformation of said hexamer within the crystal of the invention comprising the complex, (b) comparing the three-dimensional conformation as determined in step (a) with the three-dimensional structure of said hexamer within the crystal of the invention and (c) identifying compounds that inhibit the transition of the conformation of step (b) into the conformation of step (a). This protocol will also reveal conformational changes associated with binding of, for example, ssDNA that are necessary to induce the motor action of helicase. For steps (a) and (b), crystallographic methods are applicable, whereas for step (c), circular dichroism spectroscopy is particularly suited as it permits to screen various putative inhibitor compounds in a short time.

Technically, these investigations are performed with difference Fourier syntheses which require the X-ray diffraction data of a known crystal structure (RepA helicase) and X-ray diffraction data of the same crystal structure in complex with an inhibitor. The difference electron density calculated from these data shows the geometry of inhibitor-helicase interaction and provides an excellent platform for inhibitor (drug) design to optimize these interactions with the aim to obtain a better, more tightly binding inhibitor.

In addition, the present invention relates to a method of identifying an inhibitor of a bacterial helicase of hexameric structure comprising the step of identifying a compound binding under physiological conditions to the N-terminus of the protein consisting of the amino acid sequence of SEQ ID NO: 1.

This embodiment of the invention allows the inhibition of hexamer formation. As has been determined on the basis of the crystal structure of the invention, the N-terminus of the subunits of the hexameric structure are involved in the binding to the adjacent subunit. Binding of a compound to said N-terminus under physiological conditions will prevent or delay assembly of the ordered structure. This, in turn, will render the helicase inactive. In accordance with this embodiment of the invention, the term “physiological conditions” refers to conditions at pH-range and salt concentrations as found in the respective bacterial cell which are well known in the art and/or can be determined according to conventional procedures.

The term “inhibition” in accordance with the present invention means at least 50% inhibition, preferably 75% inhibition, more preferably greater 90% inhibition, most preferably more than 98% inhibition and optimally 100%. As is well known in the art, the percentage of inhibition depends on the concentration of the inhibitor.

For the above definition, concentrations of inhibitor in the range of 1 nM-10 μM are envisaged. Preferably, the concentration is in the range of 1 nM-100 nM and most preferably, the concentration is in the range of 1 nM-10 nM. All concentrations are useful for obtaining 98 or more percent inhibition.

In a preferred embodiment of the method of the present invention, said N-terminus comprises the 12 N-terminal amino acids of SEQ ID NO: 1.

In an additional preferred embodiment of the method of the present invention, said N-terminus comprises the 8 N-terminal amino acids of SEQ ID NO: 1.

The invention, furthermore, relates to a method of identifying an inhibitor of a bacterial helicase of hexameric structure comprising (a) contacting a potential inhibitor with said bacterial helicase of hexameric structure in solution and (b) determining by circular dichroism analysis whether said potential inhibitor transfers a non-ordered segment of amino acids representing a DNA binding region into an ordered structure.

Contacting said potential inhibitor with said helicase would usually be effected by incubation under suitable conditions.

Whereas the embodiment of the present invention does not directly apply to the crystals of the invention, it nevertheless relies on data obtained with the crystals of the invention. Namely, only by analyzing the three-dimensional structure of the crystals of the invention, ordered and non-ordered structures can be identified. In this way, it could be determined that the hexameric bacterial helicases comprise non-ordered structures of amino acid sequences that represent a DNA binding region. Potential inhibitors can now be tested by circular dichroism methodology for their capability to transfer a non-ordered state of such a DNA binding region into an ordered state. Transfer into an ordered structure will result in a loss or reduction of the DNA binding capacity of the helicase. Thus, a compound that will cause or contribute to said transfer would be considered as an inhibitor. Again, with the method of the invention inhibitors may be identified that can directly be formulated into pharmaceutical compositions or that can be employed as lead compounds for downstream developments.

Experimentally, inter alia the following set-up is preferred: in a circular dichroism (CD) spectrometer, a cuvette with a solution containing helicase and putative inhibitor is illuminated with right- and left-handed circularly polarized light. Since one of the two components of light is more absorbed than the other by molecules with asymmetric configuration (such as helicase), a signal (optical ellipticity) is measured that provides information about the amount of secondary structure (α-helix, β-sheet and loop conformation) of helicase. If conformational changes occur upon binding of inhibitor, these can be monitored by CD.

The present invention relates in another preferred embodiment to a method wherein said compound or potential inhibitor is a peptide, an aptamer or an antibody or derivative or fragment thereof. The term aptamer comprises nucleic acids of synthetic, semisynthetic or natural origin or derivatives thereof such as peptide nucleic acids (PNAs). PNA as well as DNA is the preferred nucleic acid to be used in accordance with the method of the invention. Derivatives of antibodies comprise, inter alia, small compounds such as scFv fragments. Fragments comprise, inter alia, Fab′ as well as F(ab)₂ or Fv fragments. As regards the employment of antibodies, monoclonal antibodies are preferred. For further guidance, it is referred to the textbook by Harlow and Lane, “Antibodies, A Laboratory Manual”, CSH Press 1988, Cold Spring Harbor, USA. A suitable assay for testing above cited molecules (putative inhibitors) would be measurement of ATPase and dsDNA unwinding activity of helicase in the presence of various amounts of these molecules.

In a preferred embodiment of the method of the present invention, said non-ordered segment corresponds to or comprises amino acids 181-200 of SEQ ID NO: 1.

In a more preferred embodiment of the method of the present invention, said non-ordered segment comprises the amino acids Arg-Gly-Ser in positions 197 to 199 of SEQ ID NO: 1.

In addition, the invention relates to a method of refining the inhibitor identified by the method of the invention comprising (a) modeling said inhibitor by peptidomimetics and (b) chemically synthesizing the modeled inhibitor. A most suitable starting point for modeling by peptidomimetrics is to test libraries of peptides of different lengths and sequences for inhibition of ATPase and ssDNA unwinding activities of RepA. In the chemical synthesis of inhibitors, the known helicase inhibitor heliquinomycin can serve as a lead compound. By determining where (i.e. to which amino acid residues) said inhibitor binds to the helicase within the crystal of the invention, the crucial amino acids for binding within the inhibitor can be identified. In following steps, the inhibitor can be optimized by chemical modification so that binding to helicase becomes tighter. Other putative inhibitors can be modeled in the same way.

In a preferred embodiment of the invention, said method further comprises the steps of (c) co-crystallizing the inhibitor as synthesized in step (b) with a bacterial helicase of hexameric structure, (d) identifying structures of said inhibitors that interact with said helicase; and (e) optimizing the inhibitor-helicase interactions by computer-aided drug design followed by chemical synthesis; or designing on the basis of the known groups interacting with helicase new analogues of said inhibitors that bind more strongly to helicase.

The invention further relates to a method of producing a pharmaceutical composition comprising an inhibitor of a hexameric bacterial helicase comprising the steps of (a) modifying an inhibitor identified by the method of the invention as a lead compound to achieve (i) modified site of action, spectrum of activity, organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmakinetic parameters (resorption, distribution, metabolism and excretion), and/or (vii) modified physico-chemical parameters (solubility, hygroscopicity, color, taste, odor, stability, state), and/or (viii) improved general specificity, organ/tissue specificity, and/or (ix) optimized application form and route by (i) esterification of carboxyl groups, or (ii) esterification of hydroxyl groups with carbon acids, or (iii) esterification of hydroxyl groups to, e.g. phosphates, pyrophosphates or sulfates or hemi succinates, or (iv) formation of pharmaceutically acceptable salts, or (v) formation of pharmaceutically acceptable complexes, or (vi) synthesis of pharmacologically active polymers, or (vii) introduction of hydrophilic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to k tales, acetales, or (xiv) N-acetylation to amides, phenylcarbamates, or (xv) synthesis of Mannich bases, imines, or (xvi) transformation of ketones or aldehydes to Schiffs bases, oximes, acetales, ketales, enolesters, oxazolidines, thiozolidines or combinations thereof; and (b) formulating the product of said modification with a pharmaceutically acceptable carrier.

The various steps recited above are generally known in the art. They include or rely on quantitative structure-action relationship (QSAR) analyses (Kubinyi, 1993), combinatorial biochemistry, classical chemistry and others (see, for example, Holzgrabe and Bechtold, 2000).

In another preferred embodiment of the various embodiments of the method of the present invention said bacterial helicase is the RepA helicase.

In an additional preferred embodiment of the method of the present invention the step of testing whether said inhibitor inhibits a eukaryotic helicase is comprised. This embodiment of the present invention is particularly advantageous because it would preclude that the inhibitor identified, refined or developed will also block the function of eukaryotic, preferably mammalian and most preferably human helicases. Advantageously, a larger number of eukaryotic helicases is tested in such assays. When administration of such inhibitors to humans is envisaged, at least one helicase of all (known) classes of human helicases should be assessed.

The invention furthermore relates to a method of identifying an inhibitor or devising a lead compound for the development of an inhibitor of a bacterial helicase of hexameric structure, said method comprising screening an at least partially randomized peptide literary, phage library or combinatorial library for peptides that fulfil the function of an inhibitor as defined herein above. Screening of said library can be effected one or more times. In this way, an inhibitor or a lead compound may be identified that has improved binding characteristics.

The libraries may preferably be

1) peptide libraries which have defined and degenerate positions in the sequence,

2) phage display libraries where a similar series of peptides is constrained in its conformation by insertion into a surface loop of a phage protein,

3) a combinatorial library whose main features are the combination of positive and negative charges and hydrophobic groups which mimic the substrate-helicase interface in the active site.

The first screens would be made for compounds that are marginally active and are able to increase the inhibition capacity 2-3 fold. In further rounds of screening, the conditions of the assay would be modified such that higher activities are needed to produce. e.g. a detectable readout of release of the radioactive label attached to the released DNA strand.

The compound identified may be further characterized for its dose response curve and by NMR, whether it binds to an active site of said hexameric helicase. It may be directly used as an active ingredient in the pharmaceutical composition to be produced in accordance with the invention. Alternatively, it may be further improved with regard to, e.g. its therapeutic features by one of the methods described herein above. The compound will be improved in its bioavailability properties such as the maximum plasma concentration and plasma elimination half-life.

The inhibitor may be further refined by improving the blocking capacity by peptidomimetics, and/or by involving phage display techniques and/or combinatorial library techniques.

All those techniques and uses thereof are known to the person skilled in the art or are immediately apparent in the light of the teachings of the present invention.

In a preferred embodiment of the method of the invention, said at least partially randomized peptide library is presented for screening by phage display. This embodiment of the invention is particularly preferred because of the high-throughput screening that has become possible using phage display screening techniques. These techniques are well known in the art and need not be described here in greater detail. For a review, it is referred to Winter et al., Annu. Rev. Immunol. 12 (1994), 433-455.

The development of a lead compound is particularly useful in cases where different mutations leading to different conformations of active centers to be influenced require the development of a variety of compounds each of which is particularly useful to influence, for example activate a specific mutant having a unique conformation.

Finally, the present invention relates to a method of producing a pharmaceutical composition comprising formulating the inhibitor identified by the method of the invention with a pharmaceutically acceptable carrier and/or diluent.

This embodiment of the invention encompasses by reference all the methods for identifying an inhibitor, refining said inhibitor or developing an inhibitor from a lead compound that are recited herein above and the various steps comprised in said methods, respectively. The pharmaceutical composition is advantageously prepared according to conventional protocols.

The pharmaceutical composition produced in accordance with the present invention may further comprise a pharmaceutically acceptable carrier and/or diluent. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg; however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. The compositions of the invention may be administered locally or systemically. Administration will generally be parenterally, e.g., intravenously. Preparations for parenteral administration include sterile aqueous or non-aqueous solutions, suspensions, and emulsions. Examples of non-aqueous solvents are propylene glycol, low molecular weight polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition.

The figures show:

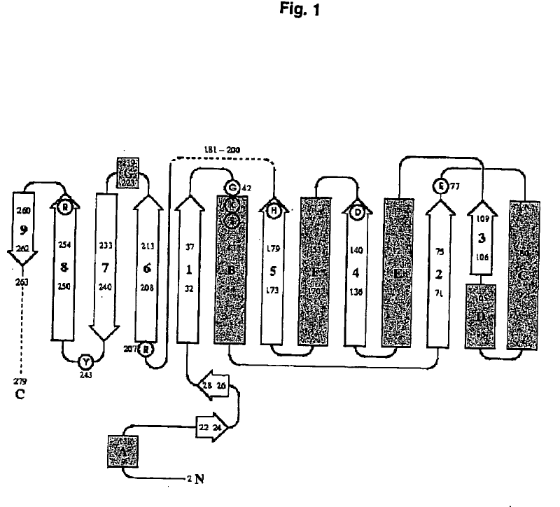

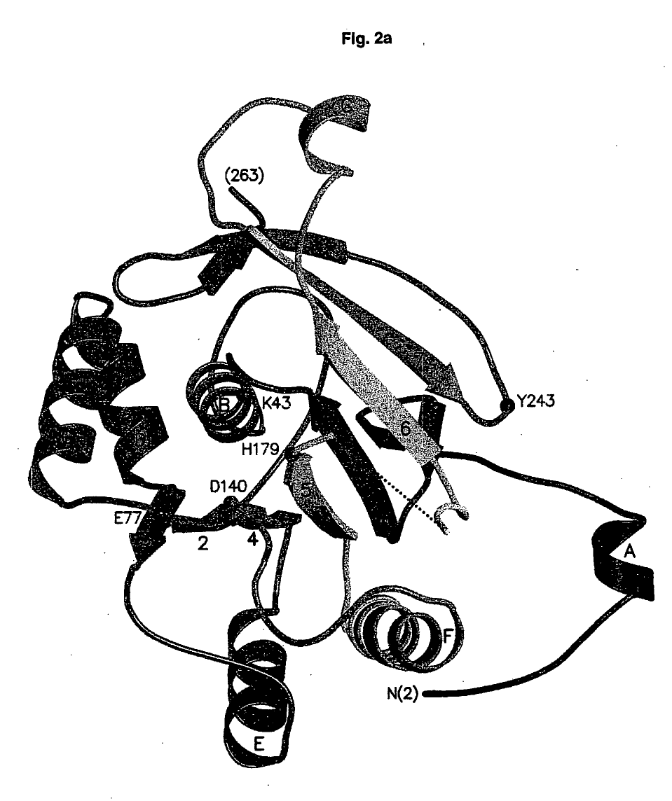

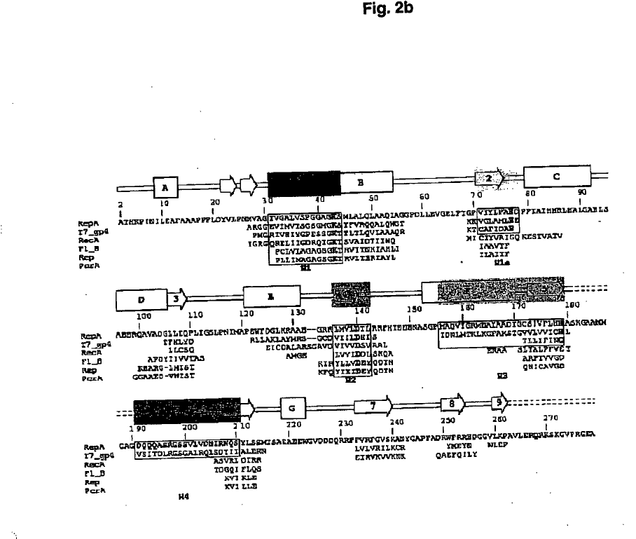

FIG. 1. Topography of RepA monomer. Strands forming α-pleated sheets drawn as arrows, α-helices as shaded rectangles; numbers denote amino acids at N- and C-termini. Met1 is missing in the polypeptide chain, the dotted lines indicate sections 181-200 and 264-279 which are not modeled as they are poorly defined in the electron density. Encircled amino acids: Gly42, Lys43, Ser44 (part of H1, see text and caption FIG. 2), Glu77 (H1a), Asp140 (H2), His179 (H3) are conserved and involved in ATP binding and hydrolysis; Arg207 is the putative “arginine finger”, Tyr243 and Arg254 sandwich the adenine base of ATP.

FIG. 2. Structure of the RepA monomer

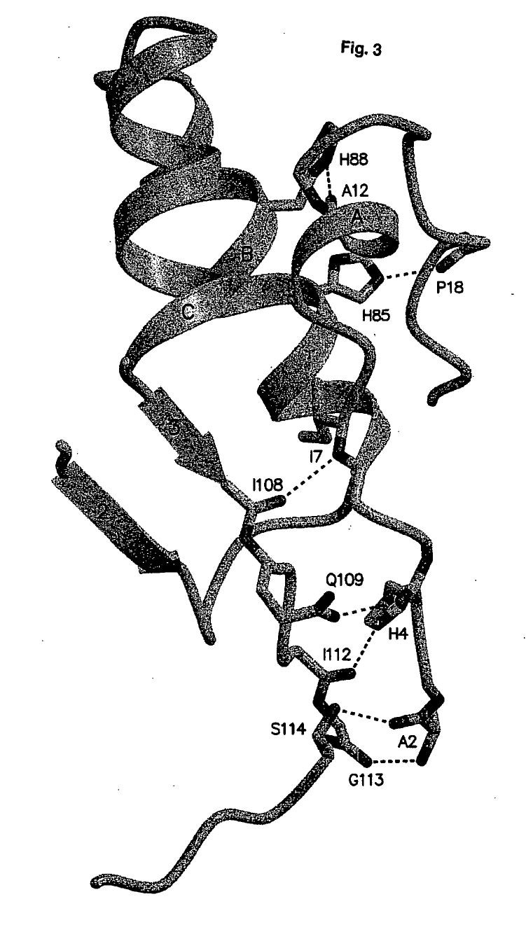

(a) The RepA monomer viewed from the center of the hexamer (see FIG. 3). N- and C-termini indicated, β-strands and α-helices labeled as in FIG. 1. The conserved helicase motifs shown in panel b are coloured accordingly. C_{α}-Positions amino acids involved in ATP binding and hydrolysis are indicated by solid colored spheres and labels. The dotted line shows the disordered segment 181-200. FIGS. 2, 3a, 4, and 5 were drawn with Molscript (Kaulis, 1991) and Raster 3D (Merrit and Bacon, 1997).

(b) Sequence comparison between RepA, the T7 helicase domain, RecA, F1-ATPase (B chain), E. coli Rep, and B. stearothermophilus PcrA: alignment of structurally equivalent residues from the output of MAPS (Lu, 1998). The secondary structure elements of RepA are shown above the aligned sequences, boxes indicate the conserved motifs of the DnaB-like helicase family H1, H1a, H2, H3, and H4.

FIG. 3. Structure of the RepA homohexamer

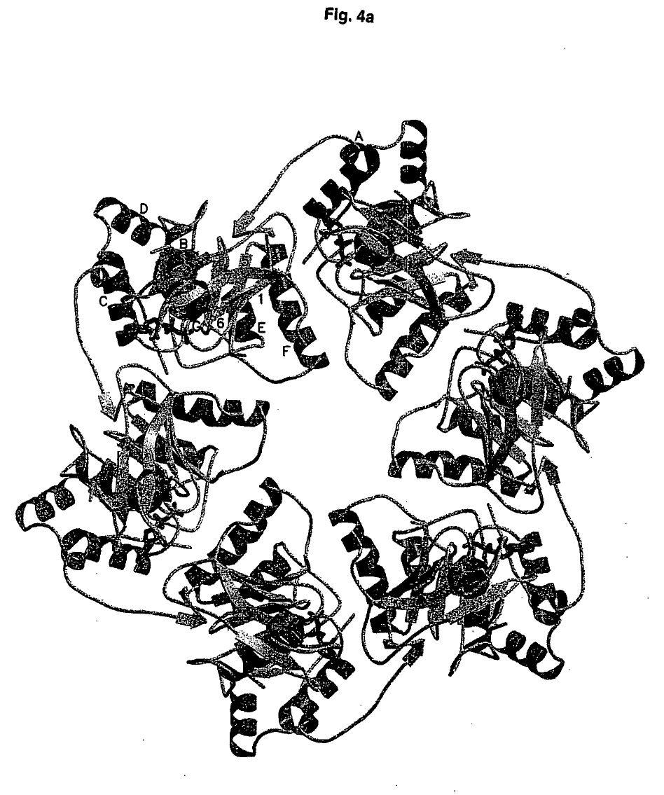

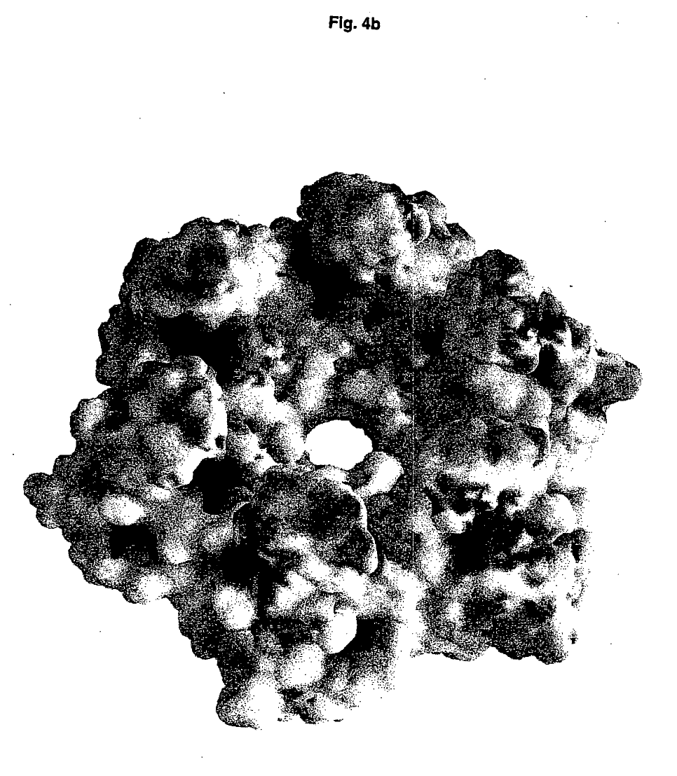

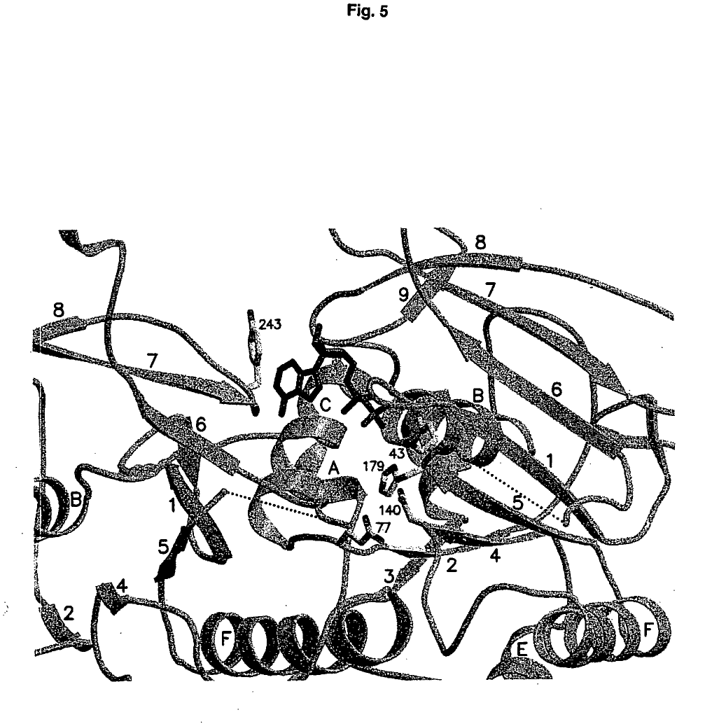

(a) The modeled complex of the pot-shaped molecule with six ATP located in clefts between the monomers (see FIG. 5) is seen from the “top”. The N-terminus of each monomer embraces the adjacent monomer as shown in FIG. 4.

(b) The solvent accessible surface of RepA without ATP was drawn with Grasp (Nicholls et al., 1991) and colored according to the electrostatic potential calculated in DelPhi (Nicholls and Honig, 1991) with full charges at all Asp, Glu, Arg, Lys and half charges at all His; positive areas are shown in blue, negative in red. The central hole has a van der Waals diameter of −17 Å, the molecule was rotated 300 about the horizontal axis for this view.

FIG. 4. Close-up view of the interactions between RepA monomers in the hexamer. The N-terminus of one monomer (yellow) is linked by a number of hydrogen bonds (dotted lines) formed by main-chain and side-chain groups to αC and the loop β3-αE of the adjacent monomer (green). The hydrogen bonds are supported by van der Waals contacts (not indicated), and there are two additional direct inter-subunit hydrogen bonds between αF of one monomer and the loop β4-αF of the adjacent monomer: Glu149 O_{ε2} . . . O_{γ} Ser153, 3.1 Å; and Arg144 N_{ε}. . . O_{ε1} Glu164, 2.8 Å (shown in FIG. 5).

FIG. 5. Section of the RepA hexamer showing the position of ATP in the cleft between two RepA monomers. ATP was modeled into the RepA hexamer according to the structurally homologous T7 helicase domain-dTTP complex, see text. ATP drawn as wire model (magenta) with adenine beween Tyr243 of one RepA monomer (yellow) and Arg254 of the neighboring subunit (green), and its triphosphate moiety located in the active site of the adjacent monomer (green) formed by Lys43, Glu77, Asp140, His179. The side chain of Arg207 (yellow) could act in trans as an “arginine finger”, contacting the γ-phosphate of ATP bound to the adjacent monomer. The dotted lines indicate the disordered loops 181-200. Additionally, the two inter-subunit contacts mentioned in the caption of FIG. 4 are shown.

The Examples illustrate the invention.

### EXAMPLE 1

### Preparation of RepA Crystals and Structural Analysis Thereof

RepA was prepared using an overproducing strain of E. coli (Scherzinger et al., 1991), purified and crystallized at pH 6.0, close to the condition of optimal helicase activity (see below) as described (Röleke et al., 1997). Crystals belong to the monoclinic space group P2₁, containing two RepA hexamers in the asymmetric unit. After many unsuccessful attempts, a heavy atom reagent was found by means of MALDI-mass spectrometry, o-chloromercuri-nitrophenol (Table 1) that bound to the single Cys172 in RepA. This derivative was used to collect MAD data which permitted to solve the crystal structure of RepA. Specifically, the following protocol for the generation of the crystals was employed. RSF1010 RepA was purified as described (Röleke et al., 1997). Crystals were grown from a RepA stock solution (38 mg/ml, 10 mM Tris-HCl, 10 mM Na₄P₂O₇) by hanging drop vapor diffusion using a precipitant solution containing 12% PEG5000-monomethylether, 3% 2-methyl-2,4-pentanediol (MPD), 150 mM NaCl, and 100 mM Na-citrate, pH6.0. For data collection under cryogenic conditions, crystals were soaked in precipitant solution containing up to 15% MPD prior to mounting in a nylon loop and flash-cooling. Multiple wavelength anomalous diffraction (MAD) data from one crystal soaked in ortho-chloromercuri-nitrophenol (CMNP, 1 mM, 24 h), and a native data set of higher resolution (Table 1) were collected at beamlines X31 and X11, respectively, of the EMBL outstation at HASYLAB/DESY, Hamburg. All data were collected at 100 K using MAR image plate detectors. The data sets were processed with DENZO/SCALEPACK (Otwinowski and Minor, 1997), and for all further calculations (except for model building and refinement), programs of the CCP4 suite (Bailey, 1994) were used. The 12 mercuri sites in the asymmetric unit (one at Cys172 of each RepA monomer) were determined from anomalous difference Patterson and Fourier syntheses. They were used to derive operators for 12-fold non-crystallographic symmetry (NCS) averaging with the program DM (Bailey, 1994), which after solvent flattening yielded an interpretable electron density map. The model was built and rebuilt using the program 0 (Jones et al., 1991); simulated annealing refinement after each major rebuilding step and final refinement of the model were performed with CNS (Brünger et al., 1998). The current model (22,659 atoms) consists of residues 2-180 and 201-263 for each of the 12 monomer chains and 591 water molecules with refined temperature factors less than 50 Å².

The structure of the globular RepA monomer (FIGS. 1, 2a) is dominated by a 9-stranded β-pleated sheet with strands 1 to 6 parallel and 7 to 9 antiparallel; amino acids 181-200 of the loop connecting β5 with β6, and the C-terminus following amino acid 263 are probably disordered and could not be modeled as the electron density was poorly defined in these sections. The α-sheet is covered by seven α-helices αA to αG and bent around αB into a semi-circle which is closed by αC, αD; helices αE, αF and αG are located at its periphery. The N-terminal segment 2-31 (Met1 is missing) projects from the globular monomer in the form of a safety pin with an extended strand (2-8), a short helix αA (9-13) and a small antiparallel β-sheet (22-24 and 26-28).

In the RepA hexamer (FIG. 3a), the N-terminal amino acids 2-18 of one monomer are linked with αC (His 85 N_{ε} and His 88 N_{δ}), and the loop following β3 (108-114) of the adjacent monomer through a number of hydrogen bonds and hydrophobic contacts formed between main-chain and side-chain groups (FIG. 4). This embracement is necessary for hexamer formation as a variant of RepA with the N-terminal 7 amino acids deleted remained monomeric under a variety of conditions (E. Lanka and G. Ziegelin, personal communication). This explains why in contrast to most other hexameric helicases, RepA does not require the presence of DNA or other cofactors to assemble into functionally active hexamers (Scherzinger et al., 1997). Additionally, there are only two inter-subunit interactions formed between the N-terminus of αF of one monomer and the long loop β4-αF of the adjacent monomer (see caption, FIG. 4), and several water-mediated hydrogen bonds (not shown). The RepA hexamer has an annular shape with a central hole, FIG. 3(a, b), as suggested previously by electron microscopy (Röleke et al., 1997). The annulus is shaped like a pot. At the outside (where the N-terminus is located), its flat bottom is formed by the right-hand side (FIG. 1) of the β-sheet (β3, β2, β4) in conjunction with helix αF; and αE forms a significant protrusion. The wall of the pot-shaped hexamer consists of the left-side of the β-sheet with antiparallel strands β6 to β9, the helices αC, αD, αG, and the disordered C-terminus. The inner bottom of the pot near the central hole is defined by the long loop β4-αF, the N-terminal part of αF, and by the disordered segment 181-200.

RepA shows optimal helicase activity at the unusually low pH 5.5 (Scherzinger et al., 1997); a similar pH-optimum has only been reported for helicase RAD3 from Saccharomyces cerevisiae (Sung et al., 1987). Since binding of RepA to substrate DNA is strongest at pH 5.5 to 6.0 and is almost abolished at pH 7.6 (Scherzinger et al., 1997), contribution of histidines is expected which are protonated at acidic pH, but aspartic and glutamic acids could be protonated as well if located in a suitable environment (Flocco and Mowbray, 1995). A surface potential plot with all His half-protonated (FIG. 3b) shows that the RepA hexamer is positively charged at its surface; prominent negatively charged areas are only found at the bottom side of the central hole (¹⁴⁸EEE¹⁵⁰ motif in loop β4-αF), near the top of the wall surrounding the pot-shaped molecule (²¹⁹EAEE²²² motif in αG), and at the outside of the hexameric ring (⁵⁹DLLEVGE⁶⁵ motif in loop αB-β2).

A DALI-search (Holm and Sander, 1993) of the protein structural database showed the topography of RepA to be comparable to those of RecA and F1-ATPase. Although in the structure-based amino acid sequence alignment of RepA with RecA and with F1-ATPase only 15% and 14%, respectively, of the structurally equivalent amino acids are identical, the Z-scores are high, 14.4 for RepA/RecA and 10.9 for RepA/F1-ATPase, suggesting close structural homology as expected (Bird et al., 1998). The structures of the monomeric hepatitis C virus RNA helicase NS3 (Yao et al., 1997), of PcrA (Subramanya et al., 1996) and of the dimeric E. coli Rep helicase (Korolev et al., 1997) are less similar to RepA with Z-scores of 3.6 (NS3) and 4.6 (PcrA). These helicases are composed of at least 2 domains of which the ATP-binding domain resembles RecA.

The structure of the RecA-ADP complex suggested that four amino acids located in the vicinity of ADP are catalytically active: Lys72, Glu96, Asp144, Gln194 (Story and Steitz, 1992). Superposition of the structures of RecA-ADP and RepA showed that these amino acids correspond within 0.9 Å to functionally equivalent amino acids in RepA highlighted by bold face in FIG. 2b: Lys43, Glu77, Asp140 and His179.

These four amino acids are all located at the C-termini of β-strands which form a typical nucleotide binding site and are identical with or are parts of conserved motifs: Lys43 on motif H1 (β1 and the loop to αβ—this is the phosphate binding or “P-loop” in F1-ATPase; Glu77 on H1a (β2); Asp140 on H2 (βb 4); His179 on H3 (part of αF including the following loop to β5).

H4 (part of the disordered segment 181-200 and of β6) apparently has no equivalent amino acid in RecA, but superposition of the NTP binding sites of RepA and of the T7 helicase domain suggests that Arg 207 of the adjacent monomer (which is located on H4 at the N-terminus of β6) could act in trans contributing its functional side chain to the ATPase site, thereby influencing the rate of ATP hydrolysis. Although its amino acid side chain is not very well defined in the electron density, Arg 207 is suitably located (FIG. 5) to play a role analogous to the “arginine finger” found in GTPase-activating proteins (Wittinghofer, 1998) and in the T7 helicase domain, although the suggested Arg 522 of T7_gp4 is not part of any conserved motif (Sawaya et al., 1999).

The RepA active site is located at the interface between two subunits, FIG. 5. If the conserved motifs of RepA and of T7 helicase domain with bound dTTP are superimposed, they closely agree (see below). On this basis, ATP was modeled into the structure of RepA. It fits snugly into the cleft formed between adjacent monomers, the triphosphate being close to the catalytic site and adenine sandwiched between Arg254 of the same and Tyr243 of the adjacent subunit. This finding is in contrast to the structures of RecA and the T7 helicase domain, where the NTP base is sandwiched between an arginine and a tyrosine of the same monomer that carries the catalytic site.

### EXAMPLE 2

### Subunit Interaction in ATP Hydrolysis

The location of functional amino acids of the catalytic site on adjacent RepA monomers suggests a mechanism of cooperativity between the subunits of the hexameric ring. The region of interest consists of the segment 179 to 207 comprising His179 at the C-terminus of β5, the disordered loop 181-200 and the following stretch of amino acids including Arg207. His179 is located near the γ-phosphate of the modeled RepA-ATP complex where it could act as a sensor changing the conformation of the following loop dependent on the presence or absence of a γ-phosphate in the binding site. This loop, which belongs partly to motif H4, contains the amino acid triplet ¹⁹⁷RGS¹⁹⁹, corresponding to the identical triplet 497-499 in the T7 helicase, where two of these amino acids, Arg497 and Gly498 are involved in DNA binding as was shown by mutational studies (Washington et al., 1996). This implies that the binding of DNA to an individual RepA monomer could be dependent on the state of the ATP binding site. Arg 207 contributes as an “arginine finger” to the catalytic site of the adjacent RepA monomer. Since only a short loop connects the DNA binding residues ¹⁹⁷RGS¹⁹⁹ with Arg207, the binding of DNA to motif H4 could in turn change the position and/or conformation of Arg207, thereby modulating the ATPase activity of the neighboring hexamer subunit.

### EXAMPLE 3

### Structural Comparison of Two Hexameric Helicases: RepA and T7 Helicase Domain

The T7 helicase domain comprises amino acids 272-566 of the helicase-primase polypeptide chain. The structure of T7 helicase domain complexed with dTTP (in the absence of Mg²⁺, so that no hydrolysis could occur) was superimposed with the structure of the RepA monomer. Although of the 121 structurally equivalent amino acids in the superposition only 26 (21%) were identical, the rms-deviation is 1.6 Å, suggesting high structural homology, especially if the five conserved motifs are considered (see FIG. 2b).

There is a salient difference, however, in the organization of subunits of RepA and of T7 helicase domain in the crystal lattice. Whereas in the former 6 subunits form a ring with 17 Å inner and 110 Å outer diameter, the subunits of T7 helicase domain are organized along the 6-fold screw axis of space group P6₁, yielding a spiral with 86 Å pitch, 120 Å outer diameter and, if projected along the 6₁ axis, a central hole of 35 Å diameter. Rearrangement of each of the subunits with 18° rotation and 10 Å translation provided a model for the ring-like T7 helicase, with the bound nucleotide located in clefts between adjacent subunits.

An interesting detail of the T7 helicase domain structure is the β bulge in motif H2 formed by an unusual cis-peptide bond following the conserved Asp424, analogous to the cis-peptide found in RecA and in the F1-ATPase. In contrast, the corresponding Asp140 in RepA is located at the C-terminal end of β4 in an exposed position, so that there is no need for a β bulge in this region.

Another notable difference between RepA and the T7 helicase domain concerns the enzymatic activity: RepA is fully functional in ATPase and helicase activities but T7 helicase domain only acts as ATPase which is stimulated by addition of ssDNA, but it is deficient in helicase activity.

### EXAMPLE 4

### Determination of the Characteristics of the Central Hole in the RepA Hexamer

The diameter of the central hole in the RepA hexamer, −17 Å, is too small to accommodate a DNA double helix in A or B conformations with diameters of ˜23 Å (Saenger, 1983). Consequently only single stranded DNA can thread through the RepA hexamer as was proposed for bacteriophage T7 helicase (Yu et al., 1996; Hacker and Johnson, 1997), if no major structural rearrangements occur upon binding of ATP and/or DNA. The dimensions of the hole are confined by the loop β4-αF (amino acids 142-152) and by the disordered segment 181-200. Both display an unusual distribution of amino acids: clustering of positive and negative charges in β4-αF: ¹⁴²LRRFHIEEEVL¹⁵² and of conformationally flexible Gly and Ala in segment 181-200: ¹⁸¹KGAAMMGAGDQQQASRGSS²⁰⁰. In RecA-ADP, the equivalent loops L1 (157-164) and L2 (195-200) are both disordered and were proposed to be involved in DNA binding (Story and Steitz, 1992), and in T7 domain a similar situation is found (Sawaya et al., 1999).

In RepA, the accumulation of 2 Arg and 3 Glu on the loop β4-αF and of 3 Gln on the disordered segment 181-200 could be essential for disruption of Watson-Crick base pairs in ds DNA as these amino acids define the central hole of the hexamer and can form bidentate hydrogen bonds with the four nucleobases A, G, T, C. This would require protonation of Glu which is possible at the pH optimum 5.5-6.0 if, as frequently observed, a carboxylate and a hydrogen bond acceptor are in suitable geometry to form a hydrogen bond, such as GluO_{ε}H . . . O═C or Glu O_{ε}H . . . :N (Flocco and Mowbray, 1995). Under these conditions, GluH⁺ and Gin can hydrogen bond in bidentate mode with functional groups of all four bases as required for non-specific interactions between helicase and DNA. (A: N6H, N1 or N7; G: N1H, O6; T: N3H, O2 or O4; C: N4H, N3), and Arg can donate two hydrogen bonds to G and C (G: O6, N7; C: O2, N3). This view is supported by the observation that of the three Glu in loop β4-αF and of the three Gln in segment 181-200 at least one Glu and one Gin each has to be present to confer enzymatic activity of RepA (E. Lanka and G. Ziegelin, personal communication).

### REFERENCES

Abrahams, J. P., Leslie, A. G., Lutter, R., and Walker, J. E. (1994). Structure at 2.8 Å resolution of F₁-ATPase from bovine heart mitochondria. Nature 370, 621-628.

Bailey, S. (1994). The CCP4 Suite—programs for protein crystallography. Acta Crystallogr. D50, 760-763.

Bárcena, M., San Martín, C., Weise, F., Ayora, S., Alonso, J. C., and Carazo, J. M. (1998). Polymorphic quaternary organisation of the Bacillus subtilis bacteriophage SPP1 replicative helicase (G40P). J. Mol. Biol. 283, 809-819

Bird, L. E., Subramanya, H. S., and Wigley, D. B. (1998). Helicases: a unifying structural theme? Current Opin. Struct. Biol. 8, 14-18.

Bowles, J. T. (1998). The evolution of aging: a new approach to an old problem of biology. Med. Hypotheses 51, 179-221.

Brünger, A. T., Adams, P. D., Clore, G. M., DeLano, W. L., Gros, P., Grosse-Kunstleve, R. W., Jiang, J.-S., Kuszewski, J., Nilges, M., Pannu, N. S., Read, R. J., Rice, L. M., Simonson, T., and Warren, G. L. (1998). Crystallography & NMR System: a new software suite for macromolecular structure determination. Acta Crystallogr. D54, 905-921.

Egelman, E. H. (1996). Homomorphous hexameric helicases: tales from the ring cycle. Structure 4, 759-762.

Ellis, N. A. (1997). DNA helicases in inherited human disorders. Curr. Opin. Genet. Dev. 7, 354-363.

Fass, D., Bogdan, C. E., and Berger, J. M. (1999). Crystal structure of the N-terminal domain of the DnaB hexameric helicase. Structure 7, 691-698.

Flocco, M. M. and Mowbray, S. L. (1995). Strange bedfellows: interactions between acidic side-chains in proteins. J. Mol. Biol. 254, 96-105.

Gorbalenya, A. E. and Koonin, E. V. (1993). Helicases: amino acid sequence comparison and structure-function relationship. Curr. Opin. Struct. Biol. 3, 419-429.

Hacker, K. J. and Johnson, K. A. (1997). A hexameric helicase encircles one DNA strand and excludes the other during DNA unwinding. Biochemistry 36, 14080-14087.

Holm, L. and Sander, C. (1993). Protein structure comparison by alignment of distance matrices. J. Mol. Biol. 233, 123-138.

Holzgrabe and Bechtold (2000). Deutsche Apotheker Zeitung 140(8), 813-823.

Ilyina, T. V., Gorgalenya, A. E., and Koonin, E. V. (1992). Organization and evolution of bacterial and bacteriophage primase-helicase systems. J. Mol. Evol. 34, 351-357.

Jones, T. A., Zou, J. Y., Cowan, S. W., and Kjeldgaard, M. (1991). Improved methods for binding protein models in electron density maps and the location of errors in these models. Acta Crystallogr. A47, 110-119.

Kaulis, P. J. (1991). MOLSCRIPT: a program to produce both detailed and schematic plots of protein structures. J. Appl. Chrystallogr. 24, 946-950.

Korolev, S., Hsieh, J., Gauss, G. H., Lohman, T. M., and Waksman, G. (1997). Major domain swiveling revealed by the crystal structures of complexes of E. coli Rep helicase bound to single-stranded DNA and ADP. Cell 90, 635-647.

Kubinyi, H. (1993). Hausch-Analysis and Related Approaches. VCH-Verlag, Weinheim.

Lohman, T. M. and Bjornson, K. P. (1996). Mechanism of helicase-catalysed DNA unwinding. Ann. Rev. Biochem. 65, 169-214.

Lu, G. (1998). An approach for multiple alignment of protein structures. Submitted to Structure

Luking, A., Stahl, U., and Schmidt, U. (1998). The protein family of RNA helicases. Crit. Rev. Biochem. Mol. Biol. 33, 259-296.

Matson, S. W. and Kaiser-Rogers, K. A. (1990). DNA helicases. Ann. Rev. Biochem. 59, 289-329.

Merrit, E. A. and Bacon, D. J. (1997). Raster 3D Photorealistic Molecular Graphics. Methods Enzymol. 277, 505-524.

Nicholls, A. and Honig, B. (1991). A rapid finite-difference algorithm, utilizing successive over-relaxation to solve the Poisson-Boltzmann equation. J. Comput. Chem. 12, 435-445.

Nicholls, A., Sharp, K. A., and Honig, B. (1991). Protein folding and association: insights from the interfacial and thermodynamic properties of hydrocarbons. Proteins Struct. Funct. Gen. 11, 281-296.

Otwinowski, Z. and Minor, W. (1997). Processing of X-ray diffraction data collected in oscillation mode. Methods Enzymol. 276, 461-472.

Röleke, D., Hoier, H., Bartsch, C., Umbach, P., Scherzinger, E., Lurz, R., and Saenger, W. (1997). Crystallization and preliminary X-ray crystallographic and electron microscopic study of a bacterial DNA helicase (RSF1010 RepA). Acta Crystallogr. D53, 213-216.

Saenger, W. (1983). Principles of nucleic acid structure. Springer Verlag New York.

Sawaya, M. R., Guo, S., Tabor, S., Richardson, C. C., and Ellenberger, T. (1999). Crystal structure of the helicase domain from the replicative helicase-primase of bacteriophage T7. Cell 99, 167-177.

Scherzinger, E., Haring, V., Lurz, R., and Otto, S. (1991). Plasmid RSF1010 DNA replication in vitro promoted by purified RSF1010 RepA, RepB and RepC proteins. Nucl. Acids Res. 19, 1203-1211.

Scherzinger, E., Ziegelin, G., Barcena, M., Carazo, J. M., Lurz, R., and Lanka, E. (1997). The RepA protein of plasmid RSF1010 is a replicative DNA helicase, J. Biol. Chem. 272, 30228-30236.

Schulz, G. E. (1992). Binding of nucleotides by proteins. Curr. Opin. Struct. Biol. 2, 61-67.

Story, R. M. and Steitz, T. A. (1992). Structure of the recA protein-ADP complex. Nature 355, 374-376.

Subramanya, H. S., Bird, L. E., Brannigan, J. A., and Wigley, D. B. (1996). Crystal structure of a DExx box helicase. Nature 384, 379-383.

Sung, P., Prakash, L., Matson, S. W., and Prakash, S. (1987). RAD3 protein of Saccharomyces cerevisiae is a DNA helicase. Proc. Natl. Acad. Sci. USA 84, 8951-8955.

Valenkar, S. S., Soultanas, P., Dillingham, M. S., Subramanya, H. S., and Wigley, D. B. (1999). Crystal structures of complexes of PcrA DNA helicase with a DNA substrate indicate an inchworm mechanism. Cell 97, 75-84.

Walker, J. E., Saraste, M., Runswick, M.-J., and Gay, N. J. (1982). Distantly related sequences in the α- and β-subunits of ATP synthase, myosin, kinases, and other ATP-requiring enzymes and a common nucleotide binding fold. EMBO J. 1, 945-951.

Washington, M. T., Rosenberg, A. H., Griffin, K., Studier, F. W., and Patel, S. S. (1996). Biochemical analysis of mutant T7 primase/helicase proteins defective in DNA binding, nucleotide hydrolysis, and the coupling of hydrolysis with DNA unwinding. J. Biol. Chem. 271, 26825-26834.

Wittinghofer, A. (1998). Signal transduction via Ras. Biol. Chem. 379, 933-937.

Yao, N. H., Hesson, T., Cable, M., Hong, Z., Kwong, A. D., Le, H. V., and Weber, P. C. (1997). Structure of the hepatitis C virus RNA helicase domain. Nat. Struct. Biol. 4, 463-467.

Yu, X., Hingorani, M. M., Patel, S. S., and Egelman, E. H. (1996). DNA is bound within the central hole to one or two of the six subunits of the T7 DNA helicase. Nature Struct. Biol. 3, 740-743.

TABLE 1
Data collection and refinement statistics

| | CMNP-1^{a} | CMNP-2 | CMNP-3 | CMNP-4 | Native |
|---|---|---|---|---|---|
| Data collection | | | | | |
| Wavelength (Å) | 1.0073 | 1.0043 | 1.0121 | 0.9999 | 0.906 |
| Resolution limit (Å) | 3.1 | 3.1 | 3.1 | 3.1 | 2.4 |
| Total observations | 292,312 | 294,164 | 290,508 | 119,093 | 377,676 |
| Unique observations | 76,800 | 76,944 | 76,705 | 44,160 | 149,192 |
| R_{sym}^{b)} | 0.095 | 0.101 | 0.085 | 0.081 | 0.034 |
| R_{sym} (last shell) | 0.265 | 0.280 | 0.253 | 0.197 | 0.117 |
| I/sigma (last shell) | 5.2 | 5.7 | 4.2 | 6.2 | 6.9 |
| Completeness | 0.998 | 0.998 | 0.996 | 0.573 | 0.946 |
| Completeness (last shell) | 0.990 | 0.984 | 0.972 | 0.605 | 0.817 |
| Model refinement | | | | | |
| R_{cryst}/R_{free}^{c)} | 0.220/0.258 | | | | |
| Resolution (Å) | 30-2.4 | | | | |
| R.m.s. deviations from | | | | | |
| stereochemical target | | | | | |
| values | | | | | |
| r.m.s.d., bond | 0.111 | | | | |
| lengths (Å) | | | | | |
| r.m.s.d., bond angles (°) | 1.56 | | | | |

^{a}CMNP, o-chloromercuri-nitrophenol
^{b)}R_{sym} = Σ|I − <I>|/ΣI, where <I> is the average intensity over symmetry equivalent reflections.
^{c)}R_{cryst} and R_{free} = Σ∥Fₒ| − |F_{c}∥/Σ|Fₒ|, where Fₒ and F_{c} are the observed and calculated structure factor amplitudes. For the calculation of R_{free}, 2% of the reflection data were selected in thin resolution shells and omitted from refinement.

1. A crystal of a hexamer of a protein consisting of the amino acid sequence of SEQ ID NO: 1, said crystal being obtainable by
(a) disrupting E. coli cells comprising a multicopy plasmid encoding Rep A helicase;
(b) removing the cell debris of the disrupted E. coli cells;
(c) applying the supernatant of said disrupted E. coli cells to an ion exchange column;
(d) eluting said polypeptide protein hexamer with a salt gradient of buffer systems A (20 mM Tris-HCl, pH 8.0, 10% glycerol, 0.1 mM EDTA, 50 mM NaCl) and B (20 mM Tris-HCl, pH 8.0, 10% glycerol, 0.1 mM EDTA, 2 mM NaCl);
(e) dialyzing the eluted protein hexamer against buffer A;
(f) applying the dialyzed protein hexamer to a heparin-agarose column;
(g) eluting the protein hexamer with a gradient of buffer systems A and B as recited in step (d);
(h) applying the eluted protein hexamer to a gel filtration column;
(i) concentrating the protein hexamer to 10 mg ml-1 in 20 mM Tris-HCl, pH 8.0, 10% glycerol, 0.1 mM EDTA and 150 mM NaCl;
(j) mixing 3 μl of concentrated protein hexamer as obtained in step (i) with 3 μl of a reservoir solution containing 20-22% (w/v) polyethyleneglycol monomethylether 5000, 2-5% (w/v) 2-methyl-2,4-pentanediol, 0.1 M citrate buffer, pH 6.0;
(k) growing crystals by
(1) transferring said mixture obtained in (j) to a siliconized microscope cover slip;
(2) turning said microscope cover slip upside down;
(3) slightly pressing said cover slip on the greased top of a well in a LINBRO® plate containing 1 ml of said reservoir solution, such that a closed system is formed in which the protein hexamer crystallizes; and
(l) soaking the crystals obtained in step (k) for 24 hours in said reservoir solution containing 1 mM of ortho-chloromercurinitrophenol.

2. A crystal of a hexamer of a protein consisting of the amino acid sequence of SEQ ID NO: 1, wherein said crystal belongs to the monoclinic space group P2₁ with unit cell constants a=104.4 Å, b=179.2 Å, c=116.3 Å, α=90.0°, β=108.8°, γ=90.0°.

3. A crystal of a further hexameric bacterial helicase, said crystal being derived from the crystal of claim 1 or 2 by employing the three-dimensional structure of RepA as a search model to locate said further helicase of comparable structure to RepA in its own crystal unit cell by rotation and translation searches.

4. A crystal comprising a hexamer of a protein as identified in claim 1 or 2 and a ligand or ligands.

5. The crystal of claim 4 wherein said ligand is a helicase substrate.

6. The crystal of claim 5 wherein said helicase substrate is a nucleoside triphosphate or an analogue thereof.

7. The crystal of claim 6 wherein said analogue is a non-cleavable analogue.

8. The crystal of claim 7 wherein said non-cleavable analogue is a non-cleavable ATP-analogue.

9. The crystal of claim 8 wherein said non-cleavable ATP-analogue is ATPγS, AMPPNP, AMPPNP, AMPPCH₂P, or ADP [AlF₄]⁻ or an analogue wherein adenine is replaced by guanine, cytosine, thymine or another heterocycle binding to said protein hexamer.

10. The crystal of any one of claims 7 to 9 wherein the ribose moiety of said non-cleavable analogue is replaced by another cyclic or non-cyclic moiety.

11. The crystal of claim 5 wherein said helicase substrate is a single-stranded DNA or a double-stranded DNA with a single-stranded overhang or an analogue thereof or heliquinomycin.

12. The crystal of claim 11 wherein said single-stranded DNA is a DNA oligonucleotide.

13. A method of identifying an inhibitor of a bacterial helicase of hexameric structure comprising
(a) determining the three-dimensional conformation of said hexamer within the crystal of any one of claims 4 to 12;
(b) comparing the three-dimensional conformation as determined in step (a) with the three-dimensional structure of said hexamer within the crystal of claim 1 or 2; and
(c) identifying compounds that inhibit the transition of the conformation of step (b) into the conformation of step (a).

14. A method of identifying an inhibitor of a bacterial helicase of hexameric structure comprising the step of identifying a compound binding under physiological conditions to the N-terminus of the protein consisting of the amino acid sequence of SEQ ID NO: 1.

15. The method of claim 14 wherein said N-terminus comprises the 12 N-terminal amino acids of SEQ ID NO: 1.

16. The method of claim 14 or 15 wherein said N-terminus comprises the 8 N-terminal amino acids of SEQ ID NO: 1.

17. A method of identifying an inhibitor of a bacterial helicase of hexamer structure comprising
(a) contacting a potential inhibitor with said bacterial helicase of hexameric structure in solution; and
(b) determining by circular dichroism analysis whether said potential inhibitor transfers a non-ordered segment of amino acids representing a DNA binding region into an ordered structure.

18. The method of claim 17 wherein said non-ordered segment is a segment corresponding to or comprising amino acids 181-200 of SEQ ID NO: 1.

19. The method of claim 17 or 18 wherein said non-ordered segment comprises the amino acids Arg-Gly-Ser in positions 197 to 199 of SEQ ID NO: 1.

20. The method of any one of claims 13 to 19 wherein said compound or potential inhibitor is a peptide, an aptamer or an antibody, derivative or fragment thereof

21. A method of refining the inhibitor identified by the method of any one of claims 13 to 20 comprising
(a) modelling said inhibitor by peptidomimetics; and
(b) chemically synthesizing the modelled inhibitor.

22. The method of claim 21 further comprising the steps of
(c) co-crystallizing the inhibitor as synthesized in step (b) with a bacterial helicase of hexameric structure;
(d) identifying structures of said inhibitors that interact with said helicase; and
(e) optimizing the inhibitor-helicase interactions by computer-aided drug design followed by chemical synthesis.

23. A method of producing a pharmaceutical composition comprising an inhibitor of a hexameric bacterial helicase comprising the steps of
(a) modifying an inhibitor identified by the method of any one of claims 13 to 20 as a lead compound to achieve
(i) modified site of action, spectrum of activity, organ specificity, and/or
(ii) improved potency, and/or
(iii) decreased toxicity (improved therapeutic index), and/or
(iv) decreased side effects, and/or
(v) modified onset of therapeutic action, duration of effect, and/or
(vi) modified pharmakinetic parameters (resorption, distribution, metabolism and excretion), and/or
(vii) modified physico-chemical parameters (solubility, hygroscopicity, color, taste, odor, stability, state), and/or
(viii) improved general specificity, organ/tissue specificity, and/or
(ix) optimized application form and route
by
(i) esterification of carboxyl groups, or
(ii) esterification of hydroxyl groups with carbon acids, or
(iii) esterification of hydroxyl groups to, e.g. phosphates, pyrophosphates or sulfates or hemi succinates, or
(iv) formation of pharmaceutically acceptable salts, or
(v) formation of pharmaceutically acceptable complexes, or
(vi) synthesis of pharmacologically active polymers, or
(vii) introduction of hydrophilic moieties, or
(viii) introduction/exchange of substituents on aromates or side chains, change of substituent pattern, or
(ix) modification by introduction of isosteric or bioisosteric moieties, or
(x) synthesis of homologous compounds, or
(xi) introduction of branched side chains, or
(xii) conversion of alkyl substituents to cyclic analogues, or
(xiii) derivatisation of hydroxyl group to ketales, acetales, or
(xiv) N-acetylation to amides, phenylcarbamates, or
(xv) synthesis of Mannich bases, imines, or
(xvi) transformation of ketones or aldehydes to Schiffs bases, oximes, acetales, ketales, enolesters, oxazolidines, thiozolidines or combinations thereof; and
(b) formulating the product of said modification with a pharmaceutically acceptable carrier.

24. The method of any one of claims 13 to 23 wherein said bacterial helicase is the RepA helicase.

25. The method of any one of claims 13 to 24 further comprising the step of testing whether said inhibitor inhibits a eukaryotic helicase.

26. A method of identifying an inhibitor or devising a lead compound for the development of an inhibitor of a bacterial helicase of hexameric structure, said method comprising screening an at least partially randomized peptide literary, phage literary or combinatorial literary for peptides that fulfil the function of an inhibitor as defined in any one of claims 13 to 25.

27. The method of claim 26 wherein said at least partially randomized peptide literary is presented for screening by phage display.

28. A method of producing a pharmaceutical composition comprising formulating the inhibitor identified or refined or developed by the method of any one of claims 13 to 27 with a pharmaceutically acceptable carrier and/or diluent.
